(19) United States
(12) Patent Application Publication
(10) Pub. No.: US 2014/0039510 A1
(43) Pub. Date: Feb. 6, 2014

(54) TPS TOOLS AND METHODS FOR THE SURGICAL PLACEMENT OF INTRAOCULAR IMPLANTS

(71) Applicants: Paul van Saarloos, Balcatta (AU); Youssef S. Wakil, Houston, TX (US)

(72) Inventors: Paul van Saarloos, Balcatta (AU); Youssef S. Wakil, Houston, TX (US)

(21) Appl. No.: 13/956,104

(22) Filed: Jul. 31, 2013

Related U.S. Application Data
(60) Provisional application No. 61/677,548, filed on Jul. 31, 2012.

(51) Int. Cl. A61F 9/007

(52) U.S. Cl. CPC A61F 9/007
USPC 606/107

(57) ABSTRACT

Provided herein are alignment systems, computer-implemented systems and methods utilizing the same for planning an optimal surgical implantation of asymmetric optics into one or both eyes of a patient and/or correcting astigmatism thereof. The methods generally comprise obtaining reference data comprising the surgical procedure plan, the reference image and the corneal topographic measurements for the patient's eye, determining an optimal placement angle of an optical zone on the cornea from the reference data and placing the asymmetric optic into the eye to match the determined optimal placement angle. Particularly, software is configured to determine the rotational difference between a reference image and a live image of the patient's eye to determine a corrected axis angle for optimal placement angle which is superimposed over a display of the live image. Also provided are non-transitory computer-readable medium and computer program products embodied with software for planning the surgical implantation.

### CROSS-REFERENCE TO RELATED APPLICATIONS

This application is a non-provisional under 35 U.S.C. §119(e) of provisional application U.S. Ser. No. 61/677,548, filed Jul. 31, 2012, now abandoned, the entirety of which is hereby incorporated by reference.

### BACKGROUND OF THE INVENTION

1. Field of the Invention

The present invention relates generally to the fields of ophthalmology and ophthalmic surgery. More specifically, the present invention relates to a measurement tool and methods for measuring and planning placement of toric ocular implants to at least minimize post-operative astigmatism or achieve desired amounts.

2. Description of the Related Art

Modern cataract surgery has embraced the benefits of placing not only spherical or aspheric intraocular lenses (IOLs) into the eye, but also toric IOLs which help to control astigmatism in the eye. The goal of toric, or astigmatic, IOLs is to correct, approximately, either the complete cylinder optics in the eye usually coming from the cornea and to maximize contrast sensitivity or to provide a desired amount of cylinder that can provide for reasonable depth of field in the eye which gives the patient a reasonable amount of far to near vision. The axis of desired cylinder can also be selected. With this higher level of sophistication of IOL designs the precise location of the axis of the IOL and overall positioning within the eye and its relation to the cornea, visual axis and/or pupil and/or other structures of the eye must be obtained to achieve the ideal outcome.

Correcting even small or moderate amounts of astigmatism, such as less than 2D, does require high precision in proper placement. With the advent of multi-focal IOLs, this is even more critical as many multi-focal designs, for example, diffractive IOLs, do not perform well with any residual astigmatism in the eye. Small errors on the level of 3-5 degrees in placing the axis of the IOL in the eye can lead to large 10-20% loss of effective correction of the toric IOL. The higher level optical performance of modern “Premium” IOLs″ and the higher expectations of the patients paying larger fees for them, require the ophthalmic surgeon to improve his/her surgical planning and techniques to obtain optimal vision performance for his patient with implantation of a toric IOL. This improved methodology also applies to other methods to correct astigmatism, including Toric ICL's or anterior chamber lenses and even corneal inlays. Correcting astigmatism in the eye with an implant generally requires placing a toric optical surface at the correct degree of rotation to cancel other sources of astigmatism in the eye such that when the optical image focuses on the retinal there is no optical cylinder, or a desired amount, if such is planned. With a toric IOL placement to replace the natural lens of the eye and as with most cataract surgeries today, the rotational placement of the IOL within the eye at the precise meridian to cancel the astigmatism from the cornea (or to leave a desired residual amount of cylinder) is planned prior to placement for an ideal outcome.

However, correctly planning the placement of toric IOLs today must overcome a series of poorly controlled measurements and marks that are all error prone and subject to changes. This results in a poorly controlled outcome in positioning the toric IOL for optimal vision correction. Given the challenges in accurately marking, measuring and placing toric IOLs, most surgeons, therefore, do not attempt the extra work required to maintain the controlled measurements necessary to adequately provide for the ideal toric IOL positioning and for this the patient's ultimate vision is sacrificed.

It is a recognized goal in the art of toric IOL surgery generally to place the IOLs toric power at the correct location in the eye to minimize or reduce to zero the astigmatism generated by the cornea. Thus, having a more direct correlation of the positioning of the IOL to the corneal topography and its optical powers would be a preferred system. The prior art is deficient in the lack of methods for measuring accurately and planning the placement of toric intraocular implants such that astigmatism in a patient's post-operative vision is corrected or minimized. The present invention fulfills this longstanding need and desire in the art.

### SUMMARY OF THE INVENTION

The present invention is directed to an alignment system for implantation of asymmetric optics into one or both eyes of a patient. The alignment system comprises means for planning a surgical procedure for implantation of the asymmetric optic, means for capturing a color reference image, means for measuring the corneal topography substantially simultaneously with capture of the color reference image, means for tangibly storing and transmitting the surgical procedure plan and the reference image to a computer, means for determining an optimal placement angle for the asymmetric optic, and means for displaying the captured reference and live images and the surgical procedure.

The present invention also is directed to a method for optimally placing an asymmetric optic into an eye of a patient during a surgical procedure. The method utilizes the alignment system described herein for obtaining reference data comprising the surgical procedure plan, the reference image and the corneal topographic measurements for the patient's eye and

determining an optimal placement angle of an optical zone on the cornea from the reference data. The asymmetric optic is placed into the eye to match the determined optimal placement angle.

The present invention is directed further to a method for correcting astigmatism in vision of a patient having cataract surgery. In step a) a reference image of the eye is captured while in step b) at substantially the same time, a corneal topography is measured to pre-determine astigmatism in a cornea of the patient's eye. In step c) an angle is determined within an optical zone of interest on the cornea of the eye for an optimal astigmatic correction. In steps d) and e) a live image of the eye is captured and is compared with the reference image to determine the difference angle between the two images. In steps f) and g) a corrected angle for the asymmetric optic on the live image is calculated from the difference angle and the corrected angle is displayed on the live image to find the corresponding angle on the eye. In step h) the implantable asymmetric optic is positioned on the eye to coincide with the corresponding angle which corrects astigmatism in the patient's vision during cataract surgery.

The present invention is directed to a related method further comprising transferring the reference image and angle for the asymmetric optic to a computer prior to step c). The present invention is directed to another related method further comprising repeating steps d) to g) at least once prior to step h). The present invention is directed to yet another related method further comprising at least minimizing post-operative residual astigmatism after step h).

The present invention is directed further still to a computer-implemented system for planning a surgical implantation of asymmetric optics into one or both eyes of a patient. The system comprises in at least one computer having a memory, a processor and at least one network connection a data module, an image and analysis module and a display module. The data module is configured to receive and transmit reference data about the patient and a pre-determined surgical plan. The image capture and analysis module is configured to receive the reference data and surgical plan, to continuously receive and digitize captured live images of the eye and locate the limbus and pupil of the eye thereon and to perform a correlation comparison between the reference image and the live images to calculate a difference in angle between them via at least a correlation algorithm to optimize a placement angle for the asymmetric optic. The display module is configured to display the live image to a computer screen and superimpose the optimized placement angle onto the live image.

The present invention is directed further still to a non-transitory computer-readable medium embodied with software for planning a surgical implantation of asymmetric optics into one or both eyes of a patient. The software when executed using at least one computer comprising the computer-implemented described herein is configured to enable instructions comprising the data module to read the reference image and axis angle relative to that image for the asymmetric optics. The software enables instructions comprising the image analysis and capture module to digitize the captured live images and to compare frame by frame the digitized images with the reference image where the comparison using correlation algorithms to calculate the rotational difference in angle between the images to determine an optimal angle. The software enables instructions comprising the display module to write the live image to a display comprising at least one of the computers and to superimpose the optimal angle onto the live image.

The present invention is directed to a related non-transitory computer-readable medium further embodied with software configured to compare the rotational difference between two images of the eye by locating the limbus and pupil of the eye in each image, sample circular sets of pixels from equivalent points on both images by taking rotational spokes from the center of the eye and sampling pixels that are the same proportion along a line between the pupil boundary and the limbus, apply a high pass filter to both circular sets, and sum the product of the two data points, one from each circle, for each rotational offset between the circular sets of data; wherein the offset angle with the highest sum is the angle difference between the two images. The present invention is directed to another related non-transitory computer-readable medium further embodied with software configured to sum the results from many circles sizes to improve the reliability of the result.

The present invention is directed further still to a computer program product, tangibly embodied in the non-transitory computer readable medium described herein.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

So that the matter in which the above-recited features, advantages and objects of the invention, as well as others which will become clear, are attained and can be understood in detail, more particular descriptions and certain embodiments of the invention briefly summarized above are illustrated in the appended drawings. These drawings form a part of the specification. It is to be noted, however, that the appended drawings illustrate preferred embodiments of the invention and therefore are not to be considered limiting in their scope.

FIG. 1. is a schematic of the components of the alignment system for implantation of asymmetric optics into one or both eyes of a patient.

FIG. 2 is a flowchart of the implantation process.

FIGS. 3A-3B are screenshots depicting the original image of the corneal topographic map (FIG. 3A), and the standard image of the eye taken at substantially the same time as the corneal topographical map (FIG. 3B).

FIG. 4 depicts the surgical plan after the details of the IOL and surgically induced change in astigmatism have been entered. The xxx line on the image marks the planned axis for the IOL.

FIG. 5 is a screenshot depicting a pre-operative plan.

FIGS. 6A-6C are screenshots depicting tools utilizable on the live image (FIG. 6A) and the use thereof on the live image (FIGS. 6B-6C).

FIG. 7 is a screenshot illustrating the pupil or limbus reference lines on the live image.

FIGS. 8A-8C are screenshots illustrating the alignment process to track the eye location on the live image.

FIGS. 9A-9D are screenshots illustrating the process of determining the axis of steep astigmatism as the eye moves in during the alignment process.

### DETAILED DESCRIPTION OF THE INVENTION

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As used herein, the term “or” in the claims refers to “and/or” unless explicitly indicated to refer to alternatives only or the alternatives are mutually exclusive, although the disclosure supports a definition that refers to only alternatives and “and/or”.

As used herein “another” or “other” may mean at least a second or more of the same or different claim element or components thereof. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the terms “computing device”, “computer” or “computer system” generally includes: a processor, a memory, at least one information storage/retrieval apparatus such as, for example, a hard drive, a disk drive or a flash drive or memory stick, or other non-transitory computer readable media or non-transitory storage device, as is known in the art, at least one input apparatus such as, for example, a keyboard, a mouse, a point and touch device, a touch screen, or a microphone; and a display structure, such as the well-known computer screen. Additionally, the computer may include one or more network connections, such as wired or wireless connections. Such a computer or computer system may include more or less than what is listed above and encompasses other electronic media, as is known in the art, for example, but not limited to tablet computers or smart devices.

As used herein, the term “toric” refers to the shape of an intraocular lens having two different curves instead of one which is utilized to correct both astigmatism and near- or farsightedness. A “toric intraocular contact lens” (ICL) refers to a very thin toric lens that is placed behind the iris and on top of the natural lens of the eye.

As used herein, the term “patient” refers to an individual or subject who has surgically received an intraocular implant and/or has surgically had placement of an intraocular implant corrected post-operatively and/or has been evaluated as a candidate for intraocular implantation. Preferably surgical procedures are or have been performed utilizing the toric calculator with correlation matching to an eye reference image presented herein.

In one embodiment of the present invention there is provided an alignment system for implantation of asymmetric optics into one or both eyes of a patient, comprising means for planning a surgical procedure for implantation of the asymmetric optic; means for capturing a color reference image; means for measuring the corneal topography substantially simultaneously with capture of the color reference image; means for tangibly storing and transmitting the surgical procedure plan and the reference image to a computer; means for determining an optimal placement angle for the asymmetric optic; and means for displaying the captured reference and live images and the surgical procedure.

In this embodiment the system may comprise a first device configured to substantially simultaneously capture a reference image of the eye and measure corneal topographic metrics of the eye; a first computing device in electronic communication with the first device and tangibly storing a first software configured to plan the surgical procedure and to receive, save and transmit input for the surgical procedure plan and the reference image and metrics; a second device configured to capture and transmit live images of the eye; a second computing device in electronic communication with the first computing device and the second device and tangibly storing a second software comprising at least a correlation algorithm configured to compare and calculate rotational or positional differences or both between the reference image and the live image and to display the same.

In another embodiment of the present invention there is provided an method for optimally placing an asymmetric optic into an eye of a patient during a surgical procedure, comprising the steps of utilizing the alignment system described supra for obtaining reference data comprising the surgical procedure plan, the reference image and the corneal topographic measurements for the patient's eye; determining an optimal placement angle of an optical zone on the cornea from the reference data; and placing the asymmetric optic into the eye to match the determined optimal placement angle.

In this embodiment the rotation of the eye during the corneal topographic measurement is substantially the same as the eye in the reference image. Also, the measured corneal topography may comprise metrics of a steep axis, a flat axis and an angle of corneal astigmatism. In addition, the asymmetric optics may comprise implantable toric intraocular lenses, implantable intraocular contact lenses or corneal inlays.

Also in this embodiment the step of determining an optimal placement angle may comprise calculating a first optimal placement angle from the corneal measurements and the rotation and position of the eye in the reference image; acquiring a series of live images of the eye prior to surgical placement of the asymmetric optic; calculating a change in one or both of a rotational difference or a positional difference between the eye in the reference image and each of the live images of the eye; and adding the calculated change to the first optimal placement angle to obtain a second corrected optimal placement angle for surgical implantation of the asymmetric optic. In addition, the step of placing the asymmetric optic into the eye may comprise displaying the second corrected optimal placement angle on the live image of the eye and aligning the asymmetric optic to coincide with an axis comprising the displayed corrected placement angle.

In yet another embodiment there is provided a method for correcting astigmatism in vision of a patient having cataract surgery, comprising the steps of a) capturing a reference image of the eye; b) measuring, at substantially the same time, a corneal topography to pre-determine astigmatism in a cornea of the patient's eye; c) determining an angle within an optical zone of interest on the cornea of the eye for an optimal astigmatic correction; d) capturing a live image of the eye; e) comparing the live image of the eye with the reference image to determine the difference angle between the two images; f) calculating, from the difference angle, a corrected angle for the asymmetric optic on the live image; g) displaying the corrected angle on the live image to find the corresponding angle on the eye; and h) positioning the implantable asymmetric optic on the eye to coincide with the corresponding angle, thereby correcting astigmatism in the patient's vision during cataract surgery.

Further to this embodiment the method may comprise transferring the reference image and angle for the asymmetric optic to a computer prior to step c). In another further embodiment the method may comprise repeating steps d) to g) at least once prior to step h). In yet another further embodiment the method may comprise at least minimizing post-operative residual astigmatism after step h). In this further embodiment the minimizing step may comprise measuring residual astigmatism after implantation, calculating a new rotation and axis for the implanted asymmetric optic and repositioning the implanted asymmetric optic to match the new rotation and axis.

In all embodiments the angle for the optimal astigmatic correction may be calculated based on metrics determined from the corneal topography relative to a limbus center of the reference image. Also in all embodiments steps e) and f) may be performed via correlation algorithms configured to calculate rotational or positional differences or both between the eye on the reference image and the eye on the live image to determine a corrected axis and to superimpose the corrected axis onto the live image. In addition, the asymmetric optics may be as described supra.

In yet another embodiment there is provided a computer-implemented system for planning a surgical implantation of asymmetric optics into one or both eyes of a patient, comprising, in at least one computer having a memory, a processor and at least one network connection, a data module configured to receive and transmit reference data about the patient and a pre-determined surgical plan; an image capture and analysis module configured to receive the reference data and surgical plan, to continuously receive and digitize captured live images of the eye and locate the limbus and pupil of the eye thereon and to perform a correlation comparison between the reference image and the live images to calculate a difference in angle between them via at least a correlation algorithm to optimize a placement angle for the asymmetric optic, and a display module configured to display the live image to a computer screen and superimpose the optimized placement angle onto the live image.

In this embodiment the reference data may comprise a plan for a surgical procedure, a reference image of the patient's eye and corneal topographic measurements obtained at substantially the same time as the reference image. Also in this embodiment the data module may be configured to input first values comprising at least spherical power, surgically induced astigmatism and incision location obtained from a plan, a captured reference image of the eye and corneal topographic measurements into user-entered fields therein and to output second values for at least the optics, for an axis of placement thereof and for residual astigmatism obtained from the first values into calculated fields comprising the data module.

In yet another embodiment there is provided a non-transitory computer-readable medium embodied with software for planning a surgical implantation of asymmetric optics into one or both eyes of a patient, said software when executed using at least one computer comprising the computer-implemented system, as described supra, is configured to enable instructions comprising the data module to read the reference image and axis angle relative to that image for the asymmetric optics; enable instructions comprising the image analysis and capture module to digitize the captured live images and to compare frame by frame the digitized images with the reference image, the comparison using correlation algorithms to calculate the rotational difference in angle between the images to determine an optimal angle; and enable instructions comprising the display module to write the live image to a display comprising at least one of the computers and to superimpose the optimal angle onto the live image.

Further to this embodiment the software when executed is further configured to compare the rotational difference between two images of the eye by locating the limbus and pupil of the eye in each image; sample circular sets of pixels from equivalent points on both images by taking rotational spokes from the center of the eye and sampling pixels that are the same proportion along a line between the pupil boundary and the limbus; apply a high pass filter to both circular sets, and sum the product of the two data points, one from each circle, for each rotational offset between the circular sets of data; wherein the offset angle with the highest sum is the angle difference between the two images. In another further embodiment the software when executed is further configured to sum the results from many circles sizes to improve the reliability of the result.

In a related embodiment there is provided a computer program product, tangibly embodied in the non-transitory computer readable medium, as described supra.

Provided herein are methods, systems and tools for measuring and planning placement of asymmetric optics, for example, but not limited to, toric ocular implants (most commonly Intra-Ocular Lenses—IOLs or Intra-Ocular Contact Lenses—ICLs) or corneal inlays in the correct axis of the patient's eye for the patient to obtain the desired correction of astigmatism for the patient's post-operative vision. Also provided herein are software applications, modules, computer readable media, and computer program products, etc. that enable a surgeon to use the toric calculator to plan a pre-operative surgical implantation of an asymmetric toric lens using computer guidance during surgery. As is known in the art, such software, modules, etc. can be tangibly stored in a computer or other electronic media, such as in a computer memory or other non-transitory media storage device, retrieved therefrom and implemented therein.

Generally, in standard measuring and placing procedures, in order to place the axis of the toric IOL in the proper meridian, the ophthalmic surgeon must generate the proper metrics and system to use on the eye or through an imaging device such as a surgical microscope to achieve such measurements to guide him during surgery in placing the IOL at the right meridian and with ideal centration and positioning to the pupil and cornea and the eye's other components including possibly, the visual axis, corneal vertex, center of limbus and/or center of pupil. The traditional procedure used to create such a metric system begins with the surgeon or a technician making a mark on the eye to determine the horizontal or 180 degree meridian as the patient is prepared for surgery. This typically involves the patient seated in front of a standard slit lamp observational microscope at which the patient is fixating on a coaxial light source. The observer determines that the patient has proper fixation and then uses a marking tool, usually blotted with an ink dye, and effectively pushes the marking tool down onto the cornea and/or the sclera of the eye to provide a “horizontal mark” for instance. This mark can be a short line or dot at the periphery of the cornea, usually at or across the limbus onto the sclera or “white of the eye”, so it can easily be observed at both the 3 o'clock and 9 o'clock positions, i.e., the 0 and 180 degree semi-meridians.

In marking the positions on the cornea or sclera a significant error is introduced as the patient's eye can move easily or rotate during the marking procedure. The technician or surgeon performing the markings can introduce many sources of error or bias in their alignment technique, etc. Once these marks are on the eye, they will represent theoretically the 180 degree or correct horizontal axis reference for the surgery. Errors in assuming that these marks are correct will be perpetuated in the process to determine the correct axis of IOL position.

Generally a surgeon uses these marks as a reference by which to measure the axis for the IOL placement using some standard caliper tools that demarcate the number of degrees from horizontal desired. There are a number of well-known and standard surgical measurement tools and methodologies useful to measure each of the 360 degrees around the eye from a reference point so that a subsequent mark can be made on the eye, for example, at 85 degrees, which represents the desired axis of final rotational placement of the IOL in the eye. Any errors in determining this 85 degree axis adds to the problem of controlling astigmatism. A toric IOL may have a corresponding mark or line such that, upon placement in the capsular bag of the eye as a replacement for the human lens, the IOL is rotated to align the mark on the IOL with the mark on the corneal limbus and sclera which denotes the final positioning of the IOL.

In general, the target axis for rotational placement of the IOL is determined so that once the IOL is placed correctly along this axis it will correct the cylinder of the cornea as desired. Most toric IOLs are designed so that there is a mark on the lens that indicates one of its principle axes, either its axis of lower power or higher optical power. Usually the axis of lower power is marked and the IOL is positioned so that axis of lower power mark and the mark on the cornea or sclera which is intended to represent the axis of cylinder power that is greatest from the cornea are coincident. The corneal axis is generally referred to as the “steep axis” of the cornea. The axis of steepest curvature of the cornea then will provide the greatest optical power in a toric cornea. Therefore, it is presumed that when the IOL is rotationally positioned so that the marks on the cornea or sclera are aligned with the correlating marks on the IOL then the toric IOL should ideally neutralize the corneal astigmatism as planned.

There are a number of critical steps in measuring this process and there are errors associated with each of these steps. Currently, there is poor correlation of the placement of the IOL to the corneal topography or toric shape and power of the cornea. The standard metric systems used today by surgeon's reference marks on the eye are assumed to be horizontal or vertical and there is no true confirmation of this assumption. With cyclotorsion of the human eye from positioning the patient in the vertical to horizontal position, as needed for surgery, there are even greater sources of error introduced and what is considered horizontal in the eye when the patient is seated is clearly not the horizontal position when the patient is supine in most patients.

In addition the purely subjective nature of the observer in applying their technique to mark the “horizontal” axis of the eye given the patients head position, the quality of the ink marks and their potential to spread or blot and even to be non-visible over the few minutes until surgery occurs can affect the process. This can occur easily as fluids, such as artificial tears and anesthetic drops, are used on the eye. Furthermore, the use of surgical measuring tools such as angular calipers that are marked in 5 or 10 degree increments also leave a great deal of error and subjectivity in their use as a surgeon tries to find a target axis within one degree of accuracy given the accuracy required to truly provide the best vision.

The present invention provides an alignment system that comprises, inter alia, a an alignment or measurement tool for implantable asymmetric optics, such as implantable toric intraocular lenses, implantable toric intraocular contact lenses or corneal inlays. The system incorporates a first topography measurement device that can measure the topography of an eye and means, such as an imaging device, to take a standard color image of the eye, either simultaneously or at substantially the same time and a first computing device having software to plan the toric surgery and save the surgery plan and reference image to a computer readable media. A second imaging device acquires live images of the eye and a second computing device, such as a computer in the operating room, comprises software to read the surgical plan and reference image off the computer-readable media, digitize a live image of the same eye during surgery and apply mathematical algorithms to calculate the rotational and/or the positional difference between the eye under the microscope and the reference image, and to display, for example, by superimposing the axis on image, the corrected axis for the IOL over a live image of the eye during surgery.

Particularly, the topography measurement tool provides a means to correlate the natural features on the eye to corneal topography or refraction/wavefront measurements, such as internal optical aberrations or other measured visual properties of the eye that may interest an ophthalmic surgeon, in planning a surgical procedure, either pre-operative or post-operative, and in providing a complete guidance system to accurately place toric or other asymmetric optics into the eye. Particularly, the measurement of corneal topography, more specifically, the steep axis of the cornea's curvature can be directly correlated to the natural features in the iris and sclera to more accurately position the toric IOL to the appropriate axis to obtain precisely the visual outcome desired.

Also, the alignment system comprises a caliper measuring system, such as, a toric caliper. As an improvement over the current standard implant planning technologies, the imaging tool and measuring system provided herein incorporate corneal topography measurements, with or without wavefront and/or aberrometry measurements, using known analog and/or digital imaging techniques and ocular measurements to directly correlate and measure the corneal topography (optionally using both anterior and posterior corneal surfaces) and, therefore, its optical powers, including astigmatism, to live images during surgery using the natural patterns on the iris and blood vessels on the sclera of a patient's eye. Previously, marks are manually made on the cornea/sclera, usually at the horizontal axis. The marks could wash off or fade during surgery, and they were large and indistinct. The directed correlative guide provides for increased precision for the surgeon to appropriately and correctly place the toric IOL. The goal of achieving a single point in time data capture incorporating corneal topography analysis, an image of the eye, and optionally, with wavefront/aberrometry analysis enables direct correlative measurements to direct surgical placement of the IOL. In practically all cases the handmade markings on the cornea/sclera have a risk of becoming indistinct.

The present invention provides computer-implemented system for planning a surgical implantation of asymmetric optics into one or both eyes of a patient. The system comprises a data module, an image capture and analysis module and a live surgery display. The data module is configured to input the pre-determined surgical plan and reference image. The image capture and analysis module continuously digitizes images from the microscope camera, locates the limbus and pupil of the eye, and performs a correlation comparison between the reference image and the microscope image to determine the difference in angle between them. It then calculates the angle for the axis of the IOL from the surgical plan and the difference in angles between the live image and the reference image. The live surgery display module writes a live image from the microscope camera on the computer screen with the most recently calculated axis for the IOL superimposed on top of the live image.

Particularly, the data module is configured to, for example, but not limited to, input into user-entered fields first values for at least IOL spherical power, surgically induced astigmatism and incision location and to output into calculated fields second values, calculated from the first inputted values, at least lens data, an axis of placement of the asymmetric optics in the one or both eyes and an expected or desired residual astigmatism. The axis of placement is relative to an image of the eye taken at the same time or close enough time so as the eye is at the same rotation as when the topography of the cornea of the eye was measured. The asymmetric optics are selected based on the calculated values.

Furthermore, the present invention comprises two particular sets of software. The first set of software is configured to enable processor-executable instructions to create the surgery plan, using the topography and other data, to get a desired axis angle for the asymmetric lens relative to a reference image. The second set of software is configured to compare the reference image with the live image from the microscope, and determines the position and/or amount the eye has rotated under the microscope compared to the reference image. It then uses this measured angle to correct the axis that the IOL is desired to be positioned at relative to the microscope image. By drawing lines over a live image display of the microscope image, the software can guide the surgeon to position the lens at the desired angle. The software can not only guide the surgeon through visual displays as described before, but could also can provide some audible sounds indicating how to turn IOL (clockwise or counter-clockwise) and possibly to indicate when IOL position is correct. The software may have the ability to automatically detect the production markings on Toric IOL's so it will know the axis of placement of the IOL and then compare to the desired axis as desired.

Thus, the present invention provides a non-transitory computer-readable medium embodied with the described software to plan a surgical implantation of asymmetric optics into one or both eyes of a patient. Also, provided is a computer program product that is tangibly embodied in the non-transitory computer readable medium. As a representative example, the computer readable medium tangibly stores instructions for execution in a computer or computing device to perform a general method of reading the reference image and axis angle relative to that image for a toric lens, digitizing the video images from the surgical microscope, comparing frame by frame the digitized images with the reference image, the comparison using correlation algorithms to calculate the difference in angle between the images.

In addition, the executable instructions may comprise comparing the rotational difference between two images of the eye. The limbus and pupil of the eye are located in each image. Circular sets of pixels are sampled from equivalent points on both images by taking rotational spokes from the center of the eye and sampling pixels that are the same proportion along the line between the pupil boundary and the limbus. A high pass filter may be applied to both circular sets, and for each rotational offset between the circular sets of data sum the product of the two data points, one from each circle. The offset angle with the highest sum is the angle difference between the two images. The executable instructions further may comprise summing the results from many circles sizes to improve the reliability of the result. For circle sizes larger than the limbus the pupil is ignored, and instead the sampled radius is the same proportion of the radius beyond the limbus in each image. Care should be taken to ensure data from eye-lids and eye lashes are excluded from the calculation if they are sampled in either image.

Furthermore, utilizing the systems, tools and software provided herein, the present invention provides methods for optimally placing asymmetric optics in an eye of a patient. A representative method comprises making reference image of the eye and at substantially the same time, measuring the corneal topography of that eye to map its metrics of a steep axis, a flat axis and an angle of corneal astigmatism. The optimal angle of an optical zone on the cornea is determined for placement of the asymmetric optic from the corneal topography measurement. During surgery a set of mathematical algorithms including a correlation algorithm is applied between the reference image of the eye and a live image of the eye, to calculate the change in angle between eyes in each image. This change in angle is added to the optimum angle determined for the Toric IOL, and this corrected angle is used to position the IOL. The result is superimposed over a live image of the eye, and the surgeon can position the IOL by viewing the IOL marks on the live image or pointing at them in a way that the instrument used to point is visible on the live screen.

Further still the present invention provides methods for correcting astigmatism in vision of a patient having cataract surgery. A representative method comprises measuring a corneal topography to pre-determine astigmatism in a cornea of the patient's eye and capturing a reference image of the eye at substantially the same time as the topography was captured. An angle is determined within an optical zone of interest on the cornea of the eye for an optimal astigmatic correction based on metrics determined from the corneal topography relative to the limbus center of the reference image for surgical placement into the eye of an implantable asymmetric optic. The reference image and angle for the asymmetric optic are transferred to a computer in the operating room and the live image of the eye captured from the operating microscope is compared with the reference image via correlation algorithms to determine the angle difference between the two images. This difference angle is used to calculate the angle for the IOL on the live image. The corrected angle is displayed or superimposed over a live image so the surgeon can find the corresponding angle on the eye and the implantable asymmetric optic is positioned to coincide with the optimal angle for the optical zone of interest.

The present invention also provides a method to further minimize or eliminate post-operative residual astigmatism. The residual astigmatism is measured after the implantation. A new rotation and axis for the implanted asymmetric optic required to minimize or to eliminate the residual astigmatism is calculated. The implanted asymmetric optic is repositioned thereby further minimizing the post-operative residual astigmatism.

As described below, the invention provides a number of advantages and uses, however such advantages and uses are not limited by such description. Embodiments of the present invention are better illustrated with reference to the Figure(s), however, such reference is not meant to limit the present invention in any fashion. The embodiments and variations described in detail herein are to be interpreted by the appended claims and equivalents thereof.

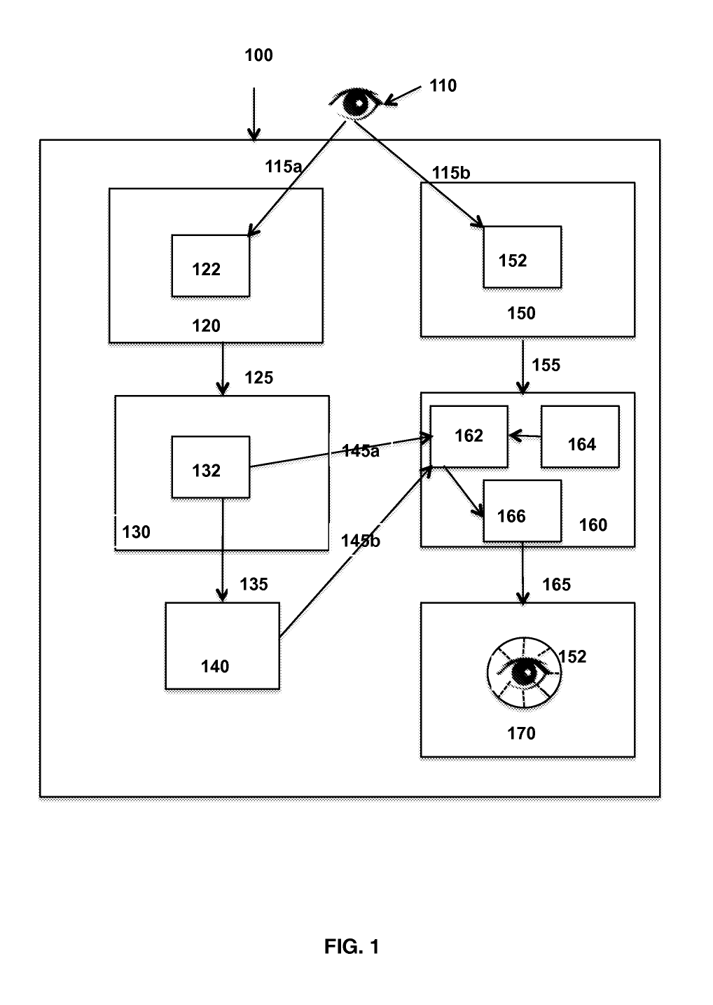

FIG. 1. is a schematic of the alignment system for implantation of asymmetric optics into one or both eyes of a patient. Generally, the system 100 comprises a first imaging device 120 configured to capture a color image of a patient's eye 110 comprising a reference image 122 and make corneal topographic measurements thereon substantially simultaneously, preferably simultaneously, at 115a. A first computing device 130, such as a computer, is in electronic communication with the first device and thereby receives the reference image and the corneal topographic measurements at 125. The first computing device comprises a data module 132 which is configured to plan the surgical procedure and to receive the reference image and corneal measurements. First values, such as at least spherical power, surgically induced astigmatism and incision location, obtained from the surgical plan, the reference image and the corneal measurements are inputted into user-entered fields comprising the data module. Second output values for at least the optics or lens, an axis of placement thereon and residual astigmatism are obtained from the first values and outputted into calculated fields in the data module. The data module is configured to tangibly store via electronic communication 135 the reference image, surgical plan, corneal measurements and associated input and output values on a non-transitory computer media storage device 140 or in the memory comprising the first computing device.

A second imaging device 150, such as a surgical microscope is configured to capture color images of the patient's eye as live images 152 at 115b and transmit the same. A second computing device 160, such as a computer in the operating room is in electronic communication with the second imaging device at 155 and receives the captured live images 164 into an image capture and analysis module 162. The second computing device receives into the image capture and analysis module the information stored in the first computing device 130 memory via electronic communication 145a or stored in the non-transitory computer-readable medium 140 via electronic communication 145b. As described supra, the image capture and analysis module comprises mathematical algorithms, particularly, at least one correlation algorithm to perform a rotational comparison frame by frame between the reference image and the live image to determine a corrected angle for optimal placement of the asymmetric optics or lens in the eye. The second computing device comprises a display module 166 configured to display the live image 152 via 165 on an interactive display screen 170, such as a computer screen, upon which the surgeon can mark positions corresponding to the optimal placement.

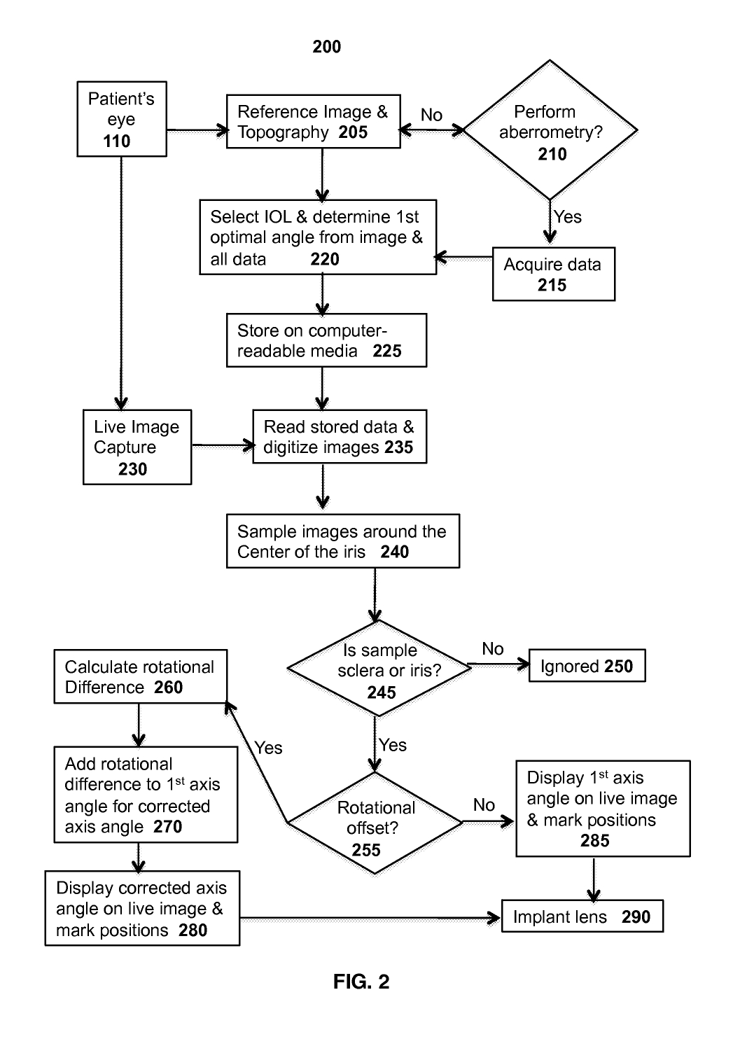

FIG. 2 is a flowchart 200 illustrating the steps in performing a toric intraocular lens (IOL) implantation with computer guidance. A patient with at least one eye 110 has been predetermined to receive a Toric IOL, most likely due to a significant degree of corneal astigmatism, and has their topography measured with a device that simultaneously records an image of the eye that includes iris and sclera details at step 205. Optionally, this can be combined with aberrometry measurements at 210 or with other diagnostic measurements, such as axial length corneal pachymetry. This can also be performed with patient seated, or supine or in any position. In a supine position the device can be held manually or by a vertical stand. If aberrometry or other diagnostic measurements are not performed then the corneal topography data, plus other data related to the patient is used to select the IOL suitable for the patient and determine the optimum angle for the Toric IOL at step 220. If aberrometry or other diagnostic measurements are performed, the data is acquired at step 215 and incorporated into step 220. The angle information and reference image of the eye are written to a computer storage media and tangibly stored thereon, along with other information that may identify the patient and be useful during surgery at step 225.

Live images of the patient's eye are captured at step 230. Software on a computer in the operating room reads the stored media and digitizes live images during surgery of the eye receiving the toric IOL at step 235. At step 240 the digitized live image is compared to the reference image and the pupil edge and limbus are found in both images. The images are sampled in circular or elliptical paths around the center of the eye. If areas of the images do not correspond to iris or sclera they are ignored at step 250. The pupil is sampled at the same proportion of distance between the pupil edge and limbus in both images, to compensate for pupil size changes of the eye and magnifications differences of the images. The sclera is sampled at the same proportion of the limbus radius beyond the limbus in both images, to compensate for magnification differences. The sampled data is filtered with a high pass digital filter.

If one or more rotational off-sets between the two images are found at 255, the two equivalent data points. one from each image on the same circular path, are multiplied together and the products summed for all data points. The rotational difference between the images, which is the rotational off-set with the highest sum, is calculated at step 260. This rotational difference is added to the axis angle that was recorded for the toric IOL position to obtain a corrected axis angle at step 270. This corrected axis angle is written over the live image displayed from the surgical microscope at step 280. If no or minimal rotational offset is found, the axis angle is displayed and marked at step 285 correspondingly to step 280 and the lens is positioned accordingly and implanted at step 290.

When a corrected axis angle is displayed at 280, the surgeon marks positions by pointing at the limbus of eye and noting the position of the instrument used to point in the live screen relative to the corrected axis positions marked on the screen. The surgeon adjusts the pointer until it coincides with the corrected axis position. The surgeon then implants the toric IOL at this axis position. The position of the IOL can be checked by pointing at the marks on the IOL and noting the position of the instrument used to point on the live video screen. The lens is then positioned to correspond to the markings and is implanted at step 290.

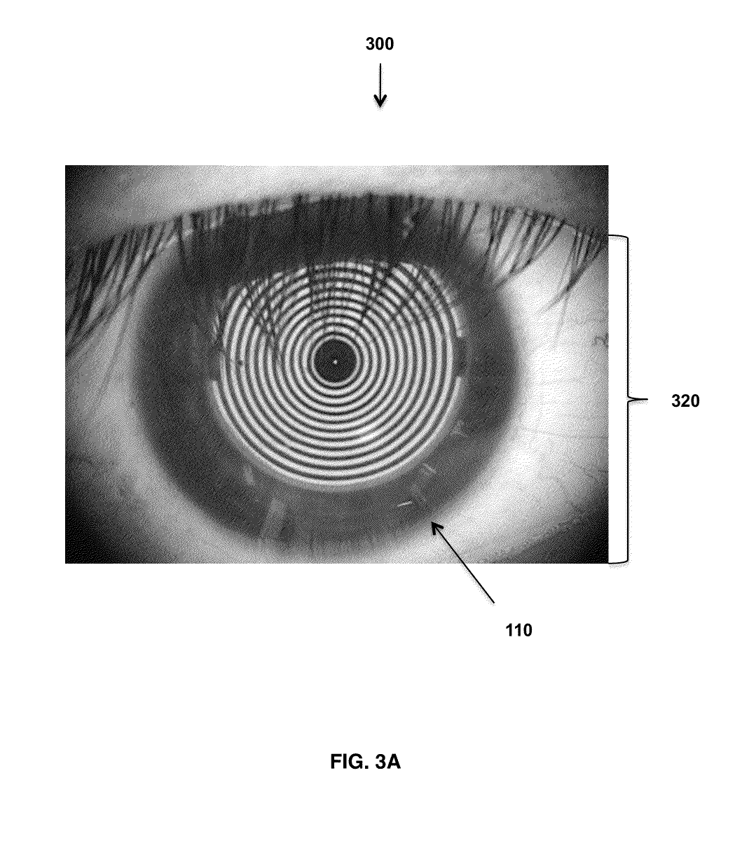

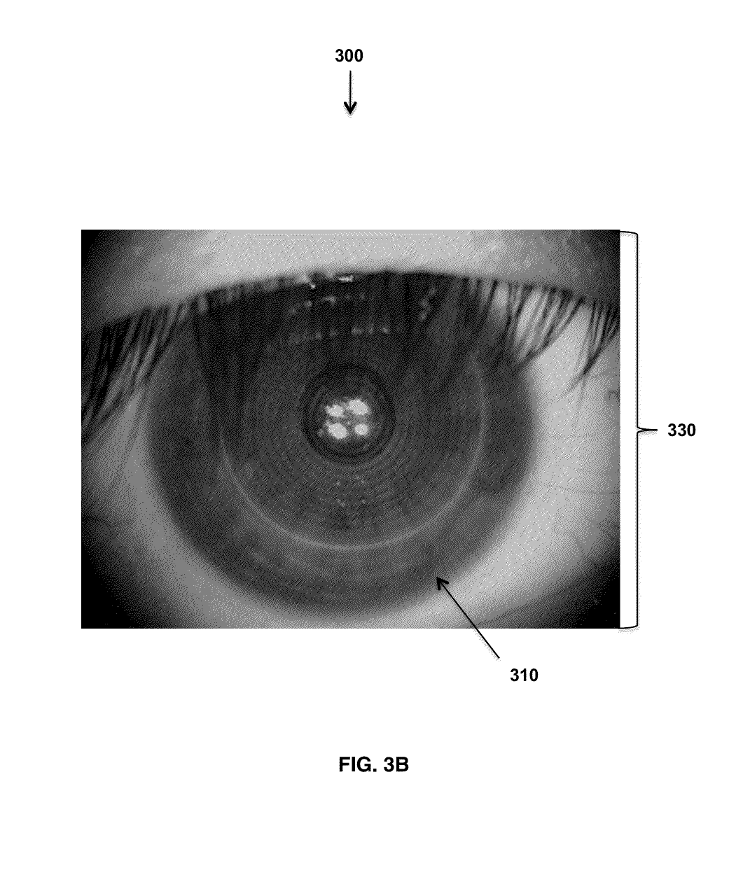

FIGS. 3A-3B show in views 300 the patient's eye as it appears during acquisition of reference data. In FIG. 3A the corneal topographic map 325 is disposed over the image 320 of the eye 310. FIG. 3B shows the image 320 which is captured as reference image 330 substantially simultaneously as corneal topographic measurements are made.

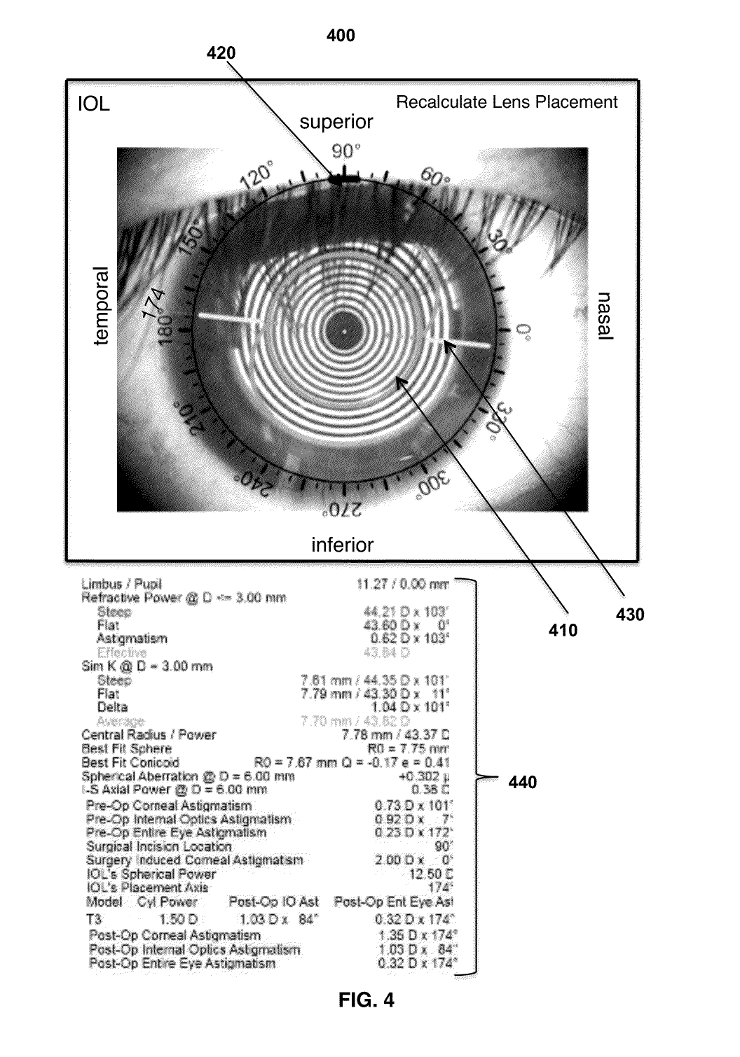

FIG. 4 illustrates the details of a surgical plan for implantation. A screenshot 400 of shows the caliper 410, the site of the surgical incision 420, and the post-operative axis 430 at 174° for the entire eye. Specific details for locating the limbus or pupil, values for corneal measurements, refractive power, the intraocular lens, aberration and for pre- and post-operative astigmatism are shown at 440.

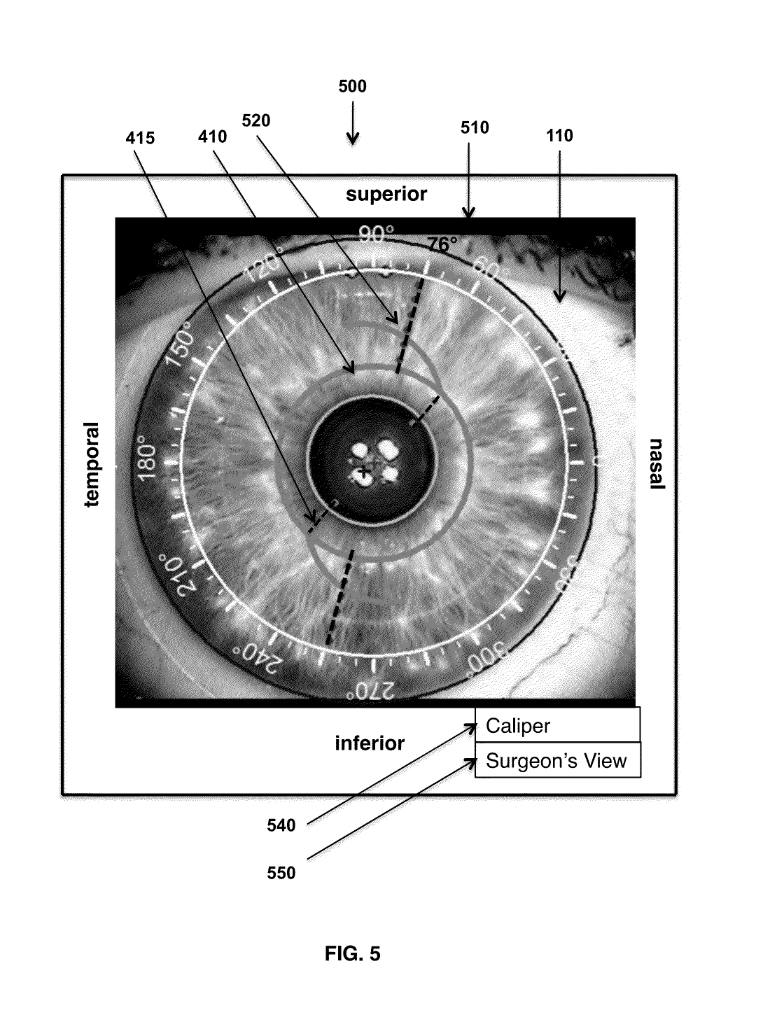

FIG. 5 is a screenshot 500 showing a pre-operative surgical plan superimposed over a live image 510 of the patient's eye 110. A toric caliper 410 is superimposed over the image. The plan displays the pre-operative axis of steep astigmatism 520 at 76°, a graphical representation of the IOL and its axis 530. The user can select to view the caliper 410 at 540 or the live camera from the microscope at surgeon's view 550.

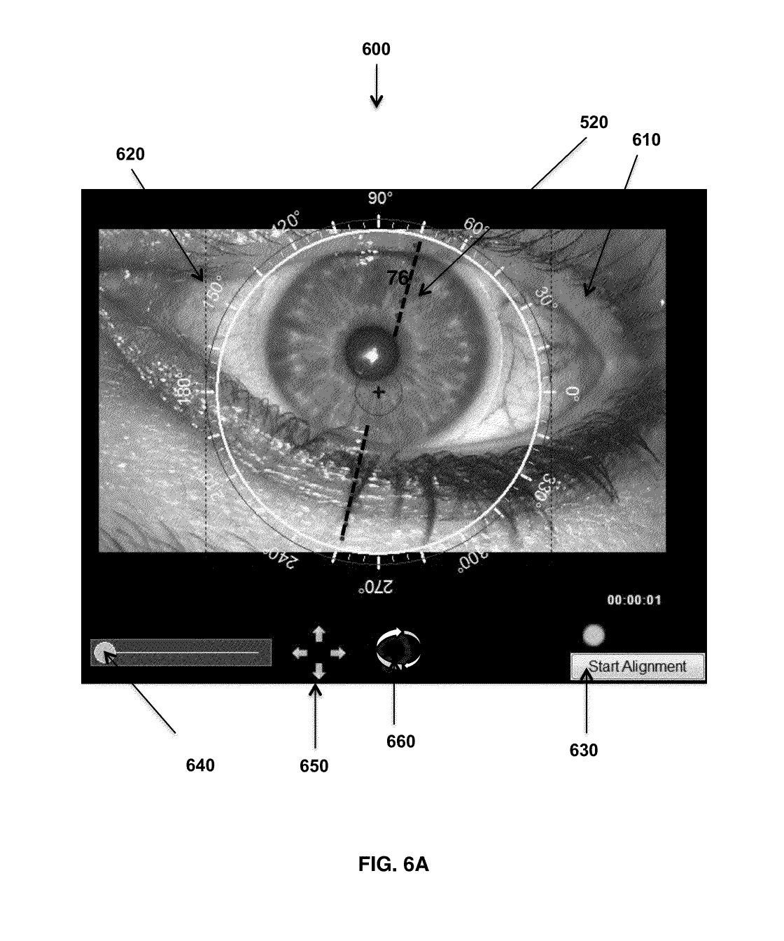

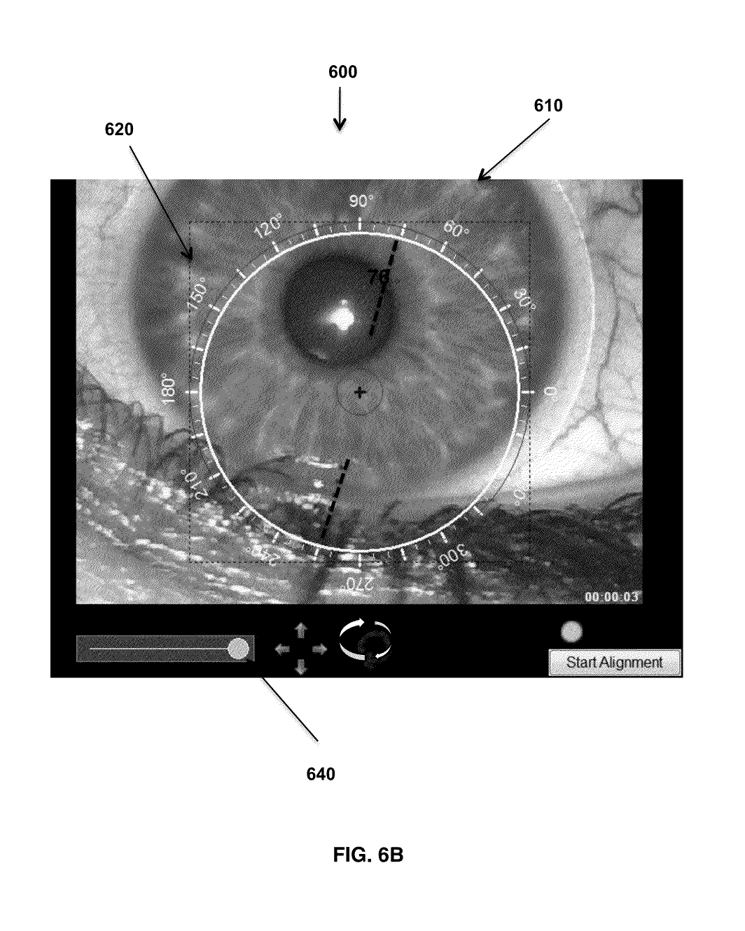

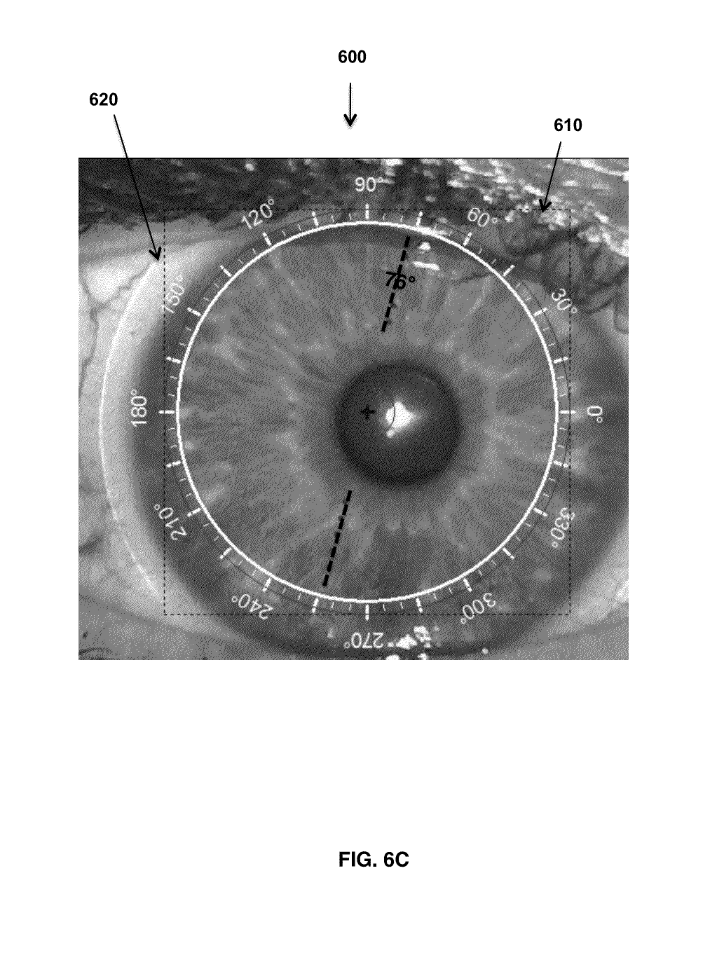

FIGS. 6A-6C illustrate on-screen tools for manipulating the size and orientation of the live image in screenshots 600. In FIG. 6A the live image 610 illustrates the pre-operative surgical plan at 620 showing the pre-operative axis of steep astigmatism 520 at 76°. The user may start the alignment process at button 630. A user may manipulate the live image by zooming in or out at using the on-screen zoom control at 640, may sequentially move the image up, down, right or left at the arrows represented by 650 or may rotate the live image at 660. In FIG. 6B the user has manipulated the live image to be the same size as the pre-operative plan 620 image using the on-screen zoom control 640. FIG. 4C illustrates the results when the user manipulated the live image 610 to be in the same orientation as the pre-operative plan 620 image using the on-screen arrow buttons 650.

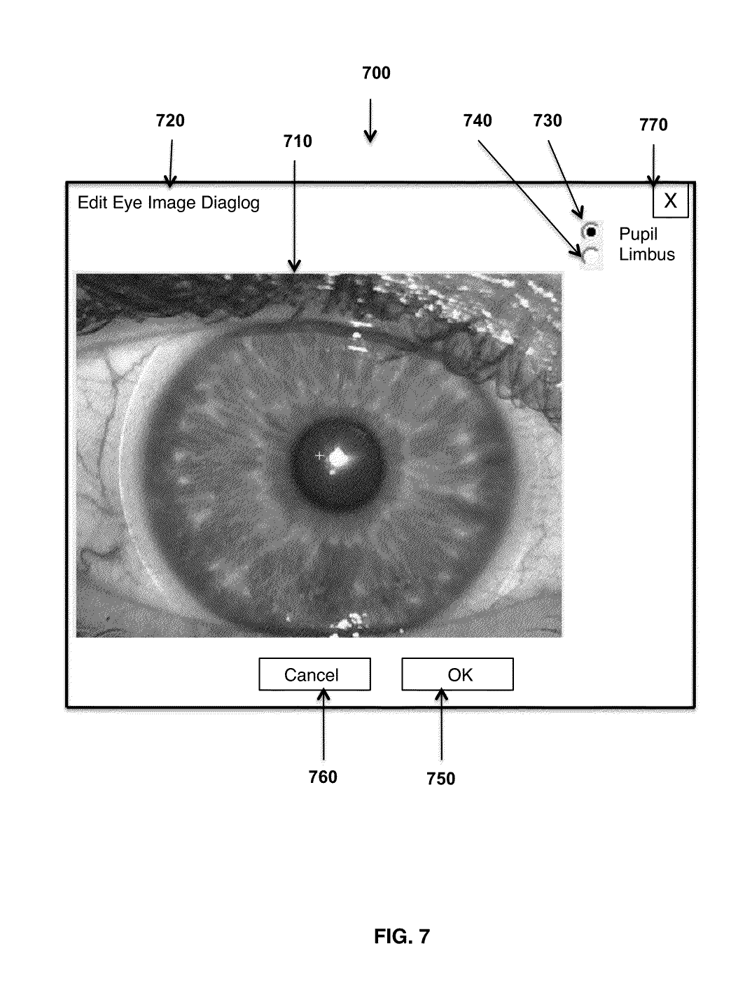

FIG. 7 is a screenshot 700 of a live image 710 depicting the Edit Eye Image Dialog 720 function. This enables the user to indicate or mark where the pupil or limbus reference lines are by first selecting the pupil button 730 or the limbus button 740. When the user is finished editing the image he may click the OK button 750 or he may cancel by clicking the cancel button 760. The user may exit at 770.

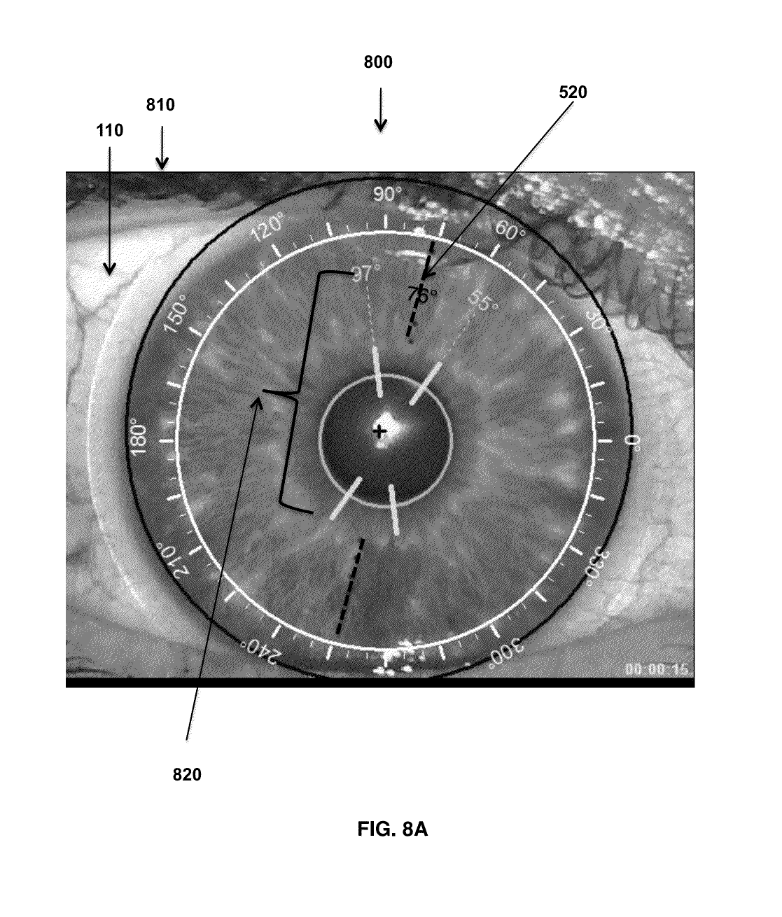

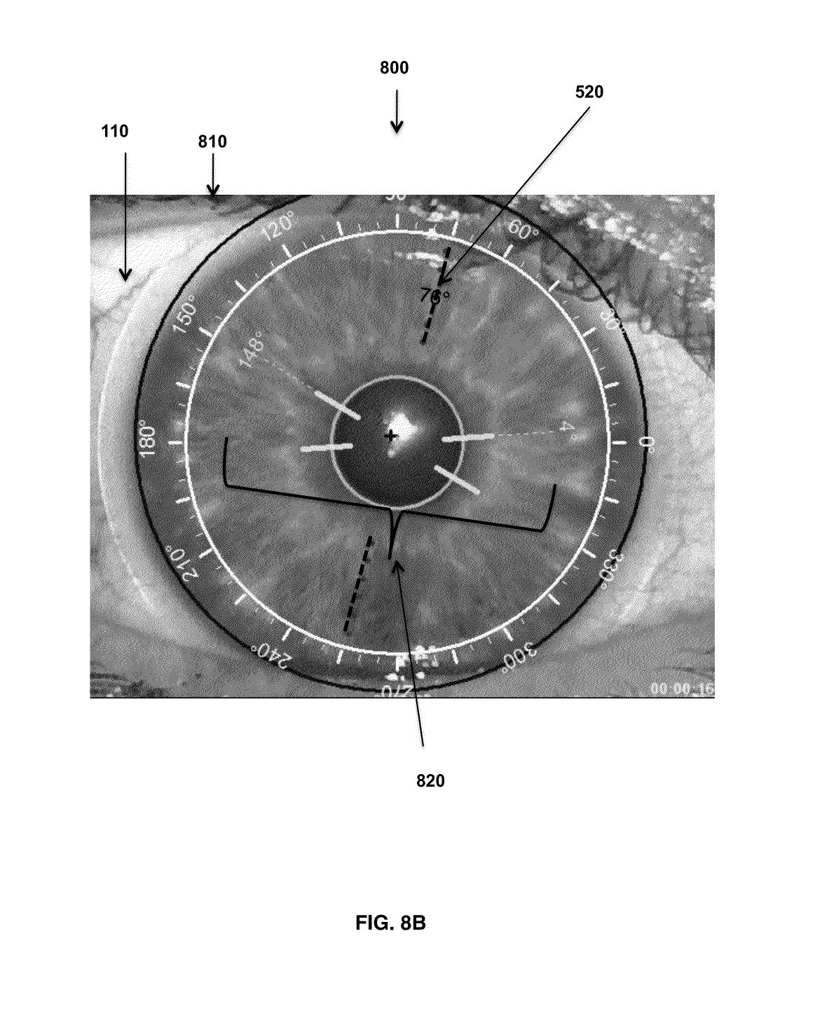

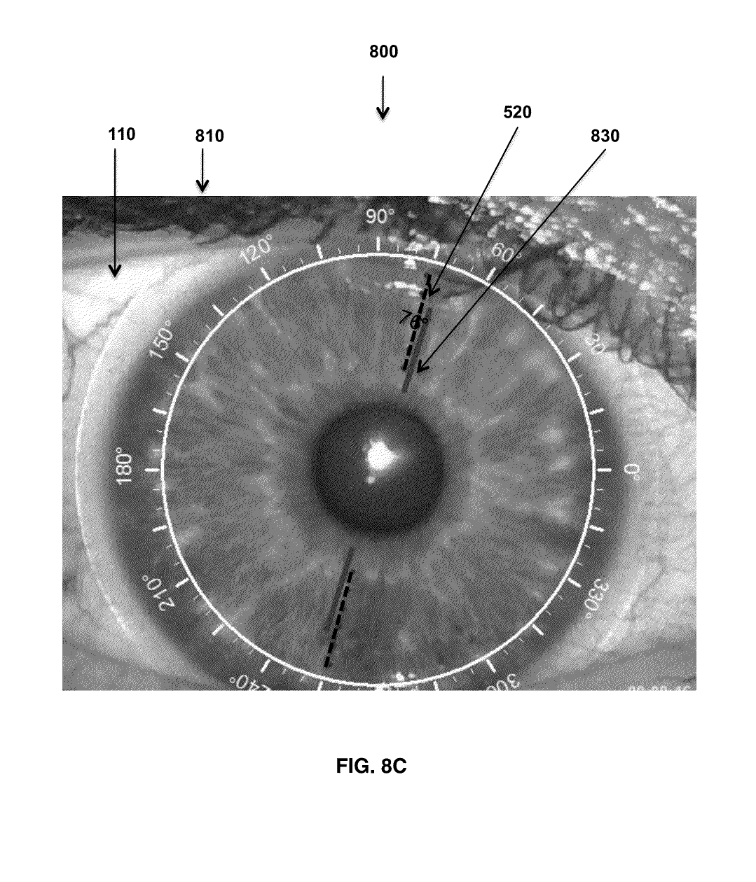

FIGS. 8A-8C illustrate the computer process of tracking the eye location in views 800. In FIG. 8A, the user has initiated the alignment process such that the computer software analyzes the live image 810 and compares it to the pre-operative plan, showing the pre-operative axis of steep astigmatism 520 at 76°, to begin tracking the eye 110 location at 820. In FIG. 8B, the program continues the image analysis and tracking of the eye location at 820. In FIG. 8C, the program finalizes analysis of the image 810 to track the eye 110 location at 830.

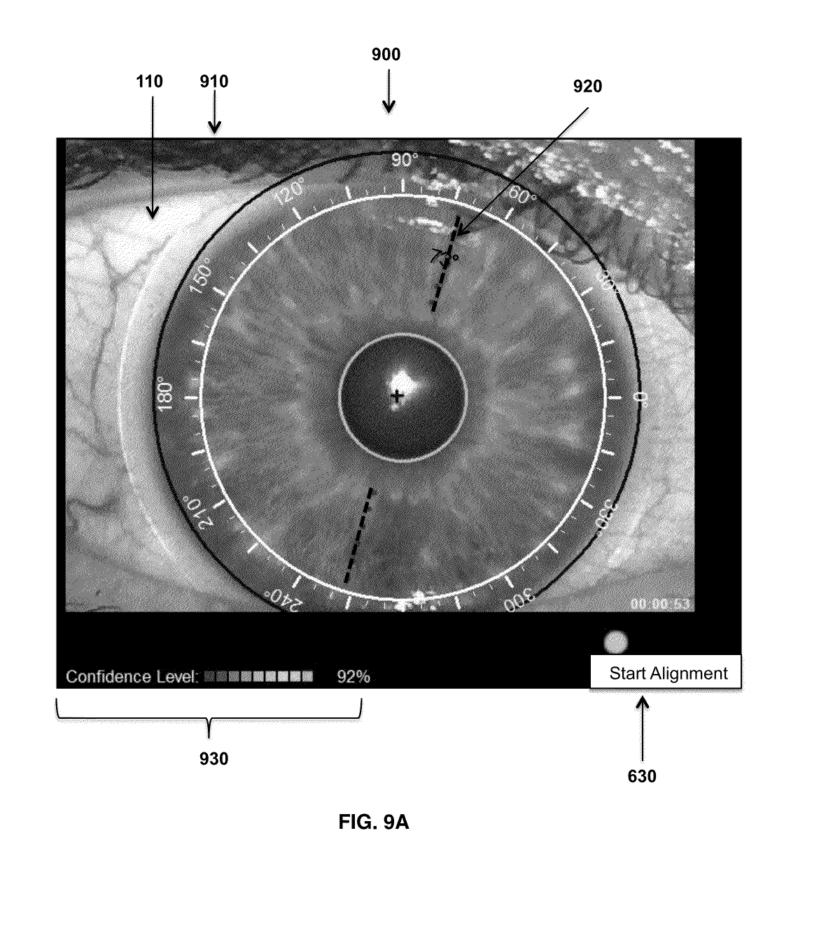

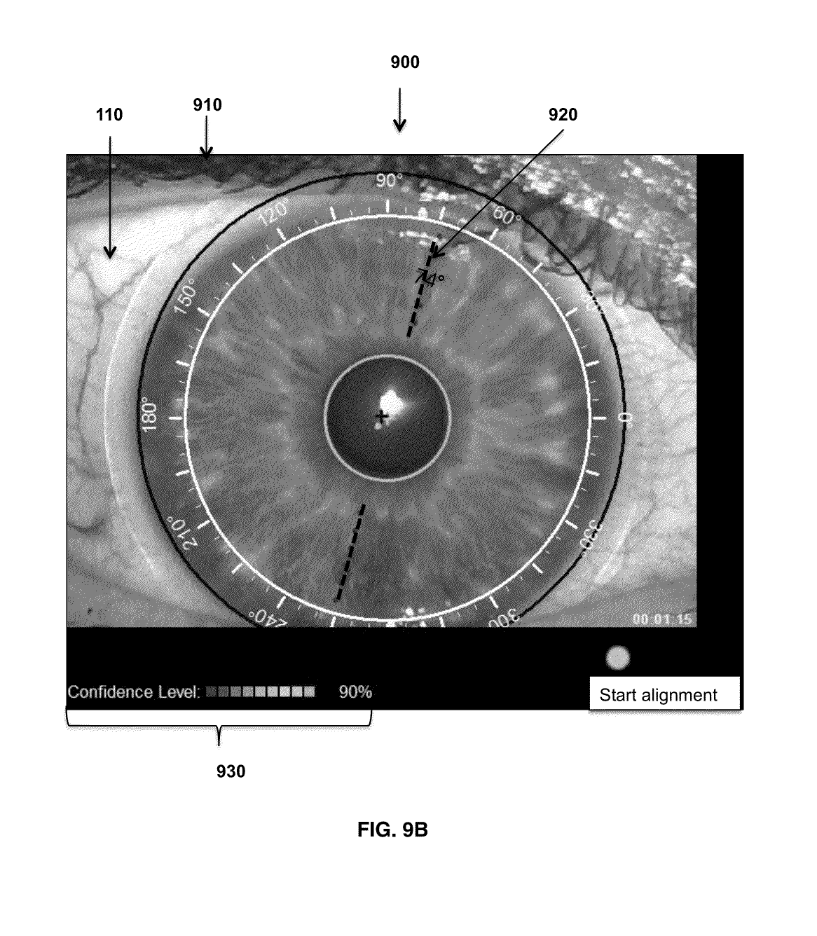

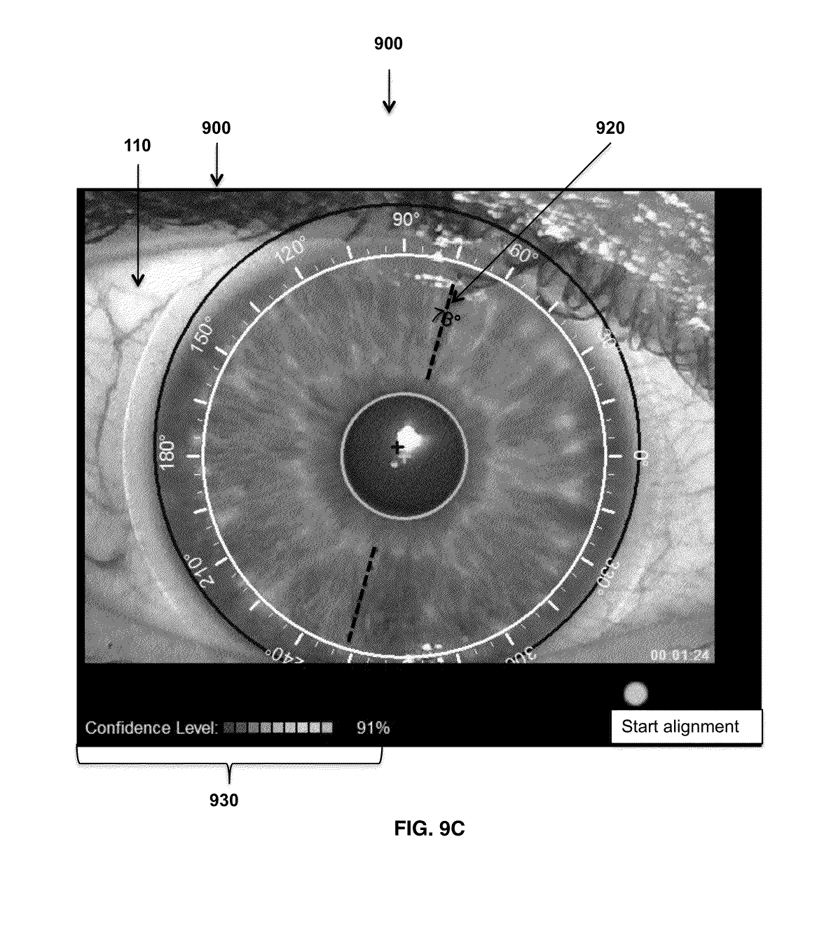

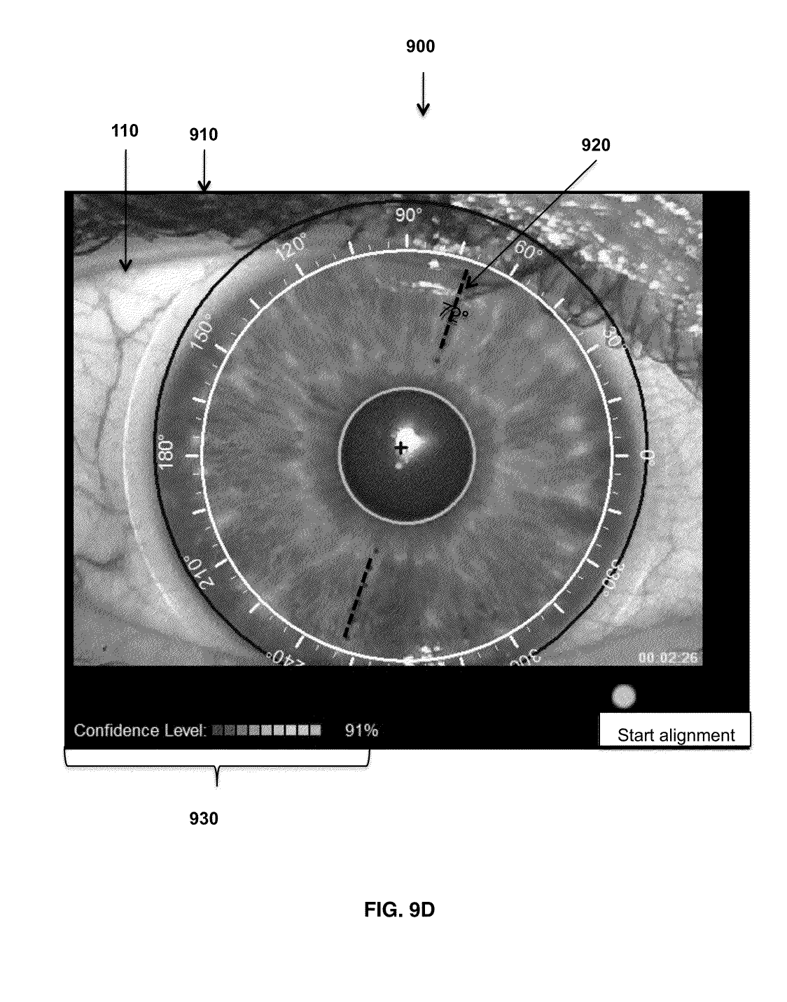

FIGS. 9A-9D illustrates the computer process of determining the actual axis of steep astigmatism in screenshots 900. In FIG. 9A the user has initiated the alignment process at 630 and has determined that the axis of steep astigmatism 920 in the live image 910 of the eye 110 is actually 73°. A confidence level of 92% is indicated at 930. In FIG. 9B the axis of steep astigmatism 920 is determined to be 74° as the live image 910 depicts the eye 110 as it continues to move. A confidence level of 92% is indicated at 930. In FIG. 9C the axis of steep astigmatism 920 is back to 73° with a confidence level of 91% at 930. The axis of steep astigmatism does not change during another iteration of alignment (not shown). In FIG. 9D the axis of steep astigmatism is 72° at 920 with slight movement. The confidence level remains 91% at 930 and has not changed since the determination in FIG. 9C.

One skilled in the art will appreciate readily that the present invention is well adapted to carry out the objects and obtain the ends and advantages mentioned, as well as those objects, ends and advantages inherent herein. The present examples, along with the methods, systems, and procedures described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. An alignment system for implantation of asymmetric optics into one or both eyes of a patient, comprising:
means for planning a surgical procedure for implantation of the asymmetric optic;
means for capturing a color reference image;
means for measuring the corneal topography substantially simultaneously with capture of the color reference image;
means for tangibly storing and transmitting the surgical procedure plan and the reference image to a computer;
means for determining an optimal placement angle for the asymmetric optic; and
means for displaying the captured reference and live images and the surgical procedure.

2. The alignment system of claim 1, wherein the system comprises:
a first device configured to substantially simultaneously capture a reference image of the eye and measure corneal topographic metrics of the eye;
a first computing device in electronic communication with the first device and tangibly storing a first software configured to plan the surgical procedure and to receive, save and transmit input for the surgical procedure plan and the reference image and metrics;
a second device configured to capture and transmit live images of the eye;
a second computing device in electronic communication with the first computing device and the second device and tangibly storing a second software comprising at least a correlation algorithm configured to compare and calculate rotational or positional differences or both between the reference image and the live image and to display the same.

3. A method for optimally placing an asymmetric optic into an eye of a patient during a surgical procedure, comprising the steps of:
utilizing the alignment system of claim 1 for:
obtaining reference data comprising the surgical procedure plan, the reference image and the corneal topographic measurements for the patient's eye;
determining an optimal placement angle of an optical zone on the cornea from the reference data; and
placing the asymmetric optic into the eye to match the determined optimal placement angle.

4. The method of claim 3, wherein the rotation of the eye during the corneal topographic measurement is substantially the same as the eye in the reference image.

5. The method of claim 3, wherein the measured corneal topography comprises metrics of a steep axis, a flat axis and an angle of corneal astigmatism.

6. The method of claim 3, wherein the step of determining an optimal placement angle comprises:
calculating a first optimal placement angle from the corneal measurements and the rotation and position of the eye in the reference image;
acquiring a series of live images of the eye prior to surgical placement of the asymmetric optic;
calculating a change in one or both of a rotational difference or a positional difference between the eye in the reference image and each of the live images of the eye; and
adding the calculated change to the first optimal placement angle to obtain a second corrected optimal placement angle for surgical implantation of the asymmetric optic.

7. The method of claim 6, wherein the step of placing the asymmetric optic into the eye comprises:
displaying the second corrected optimal placement angle on the live image of the eye; and
aligning the asymmetric optic to coincide with an axis comprising the displayed corrected placement angle.

8. The method of claim 3, wherein the asymmetric optics comprise implantable toric intraocular lenses, implantable intraocular contact lenses or corneal inlays.

9. A method for correcting astigmatism in vision of a patient having cataract surgery, comprising the steps of:
a) capturing a reference image of the eye;
b) measuring, at substantially the same time, a corneal topography to pre-determine astigmatism in a cornea of the patient's eye;
c) determining an angle within an optical zone of interest on the cornea of the eye for an optimal astigmatic correction;
d) capturing a live image of the eye;
e) comparing the live image of the eye with the reference image to determine the difference angle between the two images;
f) calculating, from the difference angle, a corrected angle for the asymmetric optic on the live image;
g) displaying the corrected angle on the live image to find the corresponding angle on the eye; and
h) positioning the implantable asymmetric optic on the eye to coincide with the corresponding angle, thereby correcting astigmatism in the patient's vision during cataract surgery.

10. The method of claim 9, further comprising the step of:
transferring the reference image and angle for the asymmetric optic to a computer prior to step c).

11. The method of claim 9, further comprising repeating steps d) to g) at least once prior to step h).

12. The method of claim 9, further comprising the step of:
at least minimizing post-operative residual astigmatism after step h).

13. The method of claim 12, wherein the minimizing step comprises:
measuring residual astigmatism after implantation;
calculating a new rotation and axis for the implanted asymmetric optic; and
repositioning the implanted asymmetric optic to match the new rotation and axis.

14. The method of claim 9, wherein the angle for the optimal astigmatic correction is calculated based on metrics determined from the corneal topography relative to a limbus center of the reference image.

15. The method of claim 9, wherein steps e) and f) are performed via correlation algorithms configured to:
calculate rotational or positional differences or both between the eye on the reference image and the eye on the live image to determine a corrected axis; and
superimpose the corrected axis onto the live image.

16. The method of claim 9, wherein the asymmetric optics comprise implantable toric intraocular lenses, implantable intraocular contact lenses or corneal inlays.

17. A computer-implemented system for planning a surgical implantation of asymmetric optics into one or both eyes of a patient, comprising, in at least one computer having a memory, a processor and at least one network connection:
a data module configured to receive and transmit reference data about the patient and a pre-determined surgical plan;
an image capture and analysis module configured to:
receive the reference data and surgical plan;
continuously receive and digitize captured live images of the eye and locate the limbus and pupil of the eye thereon; and
perform a correlation comparison between the reference image and the live images to calculate a difference in angle between them via at least a correlation algorithm to optimize a placement angle for the asymmetric optic; and
a display module configured to display the live image to a computer screen and superimpose the optimized placement angle onto the live image.

18. The computer-implemented system of claim 17, wherein the reference data comprises a plan for a surgical procedure, a reference image of the patient's eye and corneal topographic measurements obtained at substantially the same time as the reference image.

19. The computer-implemented system of claim 18, wherein the data module is configured to:
input first values comprising at least spherical power, surgically induced astigmatism and incision location obtained from a plan, a captured reference image of the eye and corneal topographic measurements into user-entered fields therein; and
output second values for at least the optics, for an axis of placement thereof and for residual astigmatism obtained from the first values into calculated fields comprising the data module.

20. A non-transitory computer-readable medium embodied with software for planning a surgical implantation of asymmetric optics into one or both eyes of a patient, said software when executed using at least one computer comprising the computer-implemented system of claim 17 is configured to:
enable instructions comprising the data module to read the reference image and axis angle relative to that image for the asymmetric optics;
enable instructions comprising the image analysis and capture module to digitize the captured live images and to compare frame by frame the digitized images with the reference image, the comparison using correlation algorithms to calculate the rotational difference in angle between the images to determine an optimal angle; and
enable instructions comprising the display module to write the live image to a display comprising at least one of the computers and to superimpose the optimal angle onto the live image.

21. The non-transitory computer-readable medium of claim 20, wherein said software when executed is further configured to:
compare the rotational difference between two images of the eye by locating the limbus and pupil of the eye in each image;
sample circular sets of pixels from equivalent points on both images by taking rotational spokes from the center of the eye and sampling pixels that are the same proportion along a line between the pupil boundary and the limbus;
apply a high pass filter to both circular sets, and
sum the product of the two data points, one from each circle, for each rotational offset between the circular sets of data; wherein the offset angle with the highest sum is the angle difference between the two images.

22. The non-transitory computer-readable medium of claim 20, wherein said software when executed is further configured to:
sum the results from many circles sizes to improve the reliability of the result.

23. A computer program product, tangibly embodied in the non-transitory computer readable medium of claim 20.
